(19) United States
(12) Patent Application Publication
(10) Pub. No.: US 2007/0110722 A1
(43) Pub. Date: May 17, 2007

(54) Methods and compositions for the Synthesis of RNA and DNA

(75) Inventors: Demian Obregon, St. Petersburg, FL (US); Mussa Ali, St. Petersburg, FL (US)

Correspondence Address: Demian Obregon, Apt. 50, 3600 E. Fletcher Ave., Tampa, FL, 33613, US

(73) Assignee: Mr. Demian Obregon, St. Petersburg, FL (US)

(21) Appl. No.: 11/164,963

(22) Filed: Dec. 12, 2005

Related U.S. Application Data
(60) Provisional application No. 60/593,121, filed on Dec. 13, 2004.

(51) Int. Cl. A61K 48/00; C12Q 1/68; C12P 19/34; C12N 15/86

(52) U.S. Cl. 424/93.2; 435/6; 435/91.2; 435/455; 435/456; 977/802

(57) ABSTRACT

Methods for the production of duplexes and single-stranded RNA and/or DNA of a desired length and sequence based on a novel template design which incorporates 2 polymerase promoters, primers, and production sequences within a single molecule are provided. This Single-stranded Template molecule design allows high-efficiency, high-yield production of single or multiple nucleic acid molecules in a single reaction vessel and thus is amenable to high-throughput automation. This Single-stranded Template molecule design also allows easy incorporation of Single-stranded Template molecules into delivery vectors for either in vitro, ex vivo, in vivo, or therapeutic application. Methods for producing Single stranded Template molecule-based RNA or DNA molecules, or hybrid molecules, in vivo and therapeutic uses for such molecules are provided. Single-stranded Template molecule kit designs are also described.

### BACKGROUND OF THE INVENTION

1. Field of Invention

Inventions related to methods and compositions for synthesizing DNA, RNA and DNA/RNA hybrids, duplexes and single-stranded RNA and/or DNA of a desired length and sequence based on the use of a Single-stranded Template molecule, in vitro, ex vivo, and in vivo.

2. Description of the Related Art

Simple and efficient methods for the generation of RNA and DNA have long been sought after in the fields of molecular biology, biotechnology and genetic engineering. Although major advances have been made in efforts to effect efficient RNA and DNA generation, more efficient methods are still needed.

Prior techniques used to concurrently generate multiple strands RNA and DNA have been rendered inefficient either by their inability to synthesize multiple nucleic-acid strands concurrently or by their necessity to implement multiple steps in order to synthesize multiple strands. Other techniques use large circular forms of nucleic acids such as plasmids, or cosmids to produce RNA or DNA. These techniques necessitate the inclusion of steps to splice in a desired production sequence, using endonucleases, in order to produce the desired nucleic acid thus adding more processing to the overall production of RNA or DNA. Similarly the use of viral constructs require the use of endonucleases to splice in the production sequence(s) of choice in order to produce the resultant nucleic acid strand of choice.

Recently, advances in nucleic acid production have spawned new approaches. These approaches are described below.

Single production sequence, single promoter sequence approach to nucleic acid strand production

One approach to the synthesis of nucleic acids is the single production sequence, single promoter sequence approach. Such technique utilizes a single promoter such as T7, or SP6, or U6 to drive the synthesis of the production sequence downstream of the promoter. The benefit of this method is the size efficiency of the construct (i.e. promoter sequence and production sequence). The drawback to this method is the inability to concurrently produce multiple strands at the same quantity, efficiency, and in the same compartment (e.g. tube, cell). The methods and compositions (i.e. Single-stranded Template) presented in this patent allows the production of two production sequences concurrently, with the same quantity of each production strand produced, with the same efficiency, and within the same compartment. Thus such constrict represent improvements over classical methods of single production sequence, single promoter sequence approaches of nucleic acid strand production. Moreover our experimental evidence suggest that during the production of two complementary strands of DNA or RNA using the Single-stranded Template methods and compositions, the complementary strands anneal to each other thus forming double stranded structures without further processing. Thus these methods and compositions are ideal for the production of large quantities of double stranded RNA or DNA without separate steps for both the sense and antisense strand production processes. Moreover since the Single-stranded Template produced strands bind to each other while being produced these methods and compositions also allow for increased efficiency by allowing the user to omit classical annealing steps that are typically necessary to anneal separately produced sense and antisense nucleic acid strands. Additionally, because two different nucleic acid strands can be produced, the construct allows for the production of molecules such as ribozymes or deoxyribozymes that can act on the product of the other production template or on a separate nucleic acid strand to allow for complex modifications to either product strand or other molecular target (e.g. cellular mRNA) or allow a layer of regulation or modulation to be added to the production of the nucleic acids. Also because there are two production sequences RNA/DNA hybrid Single-stranded Template molecules allow for RNA and DNA to be synthesized at the same time, with the same efficiency and quantity if expressed under similar promoters utilizing equivalent activities of polymerase enzymes. Moreover the design of the Single-stranded Template molecule allows for an easy incorporation into other vectors (e.g. plasmids, cosmids, bacteriophages, viruses, extrachromosomal arrays, artificial chromosomes) either for its production by the vector, or for its integration and use within the vector for the production of nucleic acid strands (i.e. RNA, DNA, ribozymes, deoxyribozymes).

Plasmid, cosmid, bacteriophage or viral approaches to the production of nucleic acid strands.

Other methods for the production of RNA or DNA utilize large circular fragments of DNA such as plasmid, cosmids, bacteriophages, or viruses. Plasmids are typically circular double stranded DNA molecules that can contain numerous production sequences. Cosmids are a type of plasmid constructed by the insertion of cos sequences enabling them to be packaged into λ phage particles in vitro. The advantages of plasmids and cosmids include the ability to construct multiple expression regions capable of producing various production sequence products concurrently. The disadvantages of plasmids and cosmids include size, complexity of production, and inefficiency in modification. Plasmids, cosmids and other vectors often require the use of endonucleases in order to splice in production sequences of choice. The Single-stranded Template design and method allows for smaller number of nucleotides to be used in the construction of the molecule and thus allows for more efficient production, modification, and also allows for more efficient transfection efficiencies. Moreover, due to its small size the Single-stranded Template can be integrated as mentioned above into other vectors for delivery or regulation. Viral and bacteriophage vectors have similar advantages and disadvantages as the aforementioned plasmids and cosmids, however their ability to effect cellular delivery of nucleic acids makes these vectors extremely attractive to genetic engineers. Again, the Single-stranded Template design and method allows for smaller number of nucleotides to be used in the construction of the molecule and thus allows for more efficient production, modification, and also allows for safer use versus many viruses and bacteriophages.

### SUMMARY OF INVENTION

Methods for the production of duplexes and single-stranded RNA and/or DNA of a desired length and sequence based on a novel template design which incorporates 2 polymerase promoters, primers, and production sequences within a single molecule are provided. This Single-stranded Template molecule design allows high-efficiency, high-yield production of single or multiple nucleic acid molecules in a single reaction vessel and thus is amenable to high-throughput automation. This single molecule design also allows easy incorporation of Single-stranded Template molecules into delivery vectors for either in vitro, ex vivo, in vivo, or therapeutic application. Methods for producing Single-stranded Template molecule-based RNA or DNA molecules, or hybrid molecules and therapeutic uses for such molecules are provided. Single-stranded Template molecule kit designs are also described.

### DRAWING FIGURES

FIG. 1 Basic design of completely folded, functional Single-stranded Template molecule

FIG. 1A Illustration showing two Single-stranded Template molecules linked via linkers

FIG. 1B Basic design of functional Single-stranded Template molecule showing details of each functional domain

FIG. 2 is a flowchart describing an example of the production and use of a Single-stranded Template molecule.

FIG. 3 are digital microscopic pictures of COS7 cells transfected with Single-stranded Template produced small interfering RNA targeting green fluorescent protein, or a non-targeting control. This figure illustrates the ability of the Single-stranded Template molecule to produce functional nucleic acid strands for use in techniques such as RNA interference.

### REFERENCE NUMERALS IN DRAWINGS

6 First linker
8 First production sequence
10 First promoter complement sequence
12 First loop sequence
14 First promoter sequence
16 Spacer sequence
18 Second promoter sequence
20 Production complement sequence
22 Second loop sequence
23 Second production sequence
24 Second promoter complement sequence
26 Second linker

### DETAILED DESCRIPTION OF INVENTION

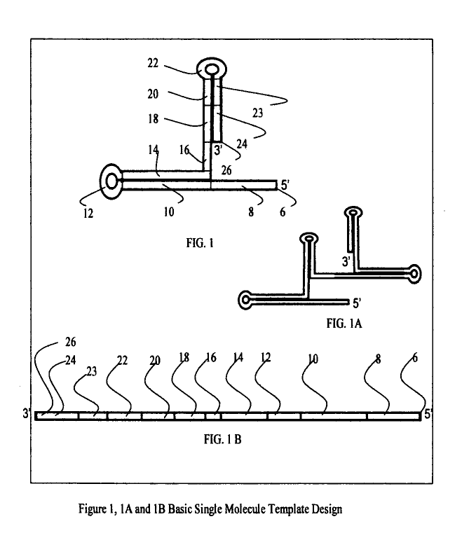

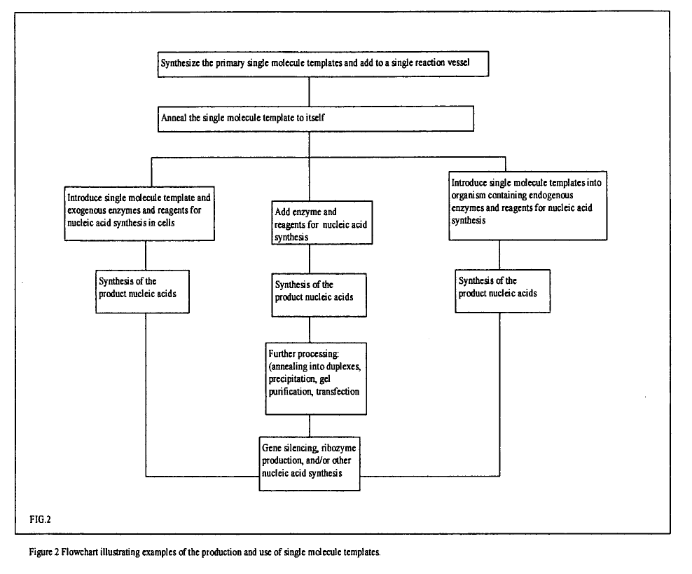

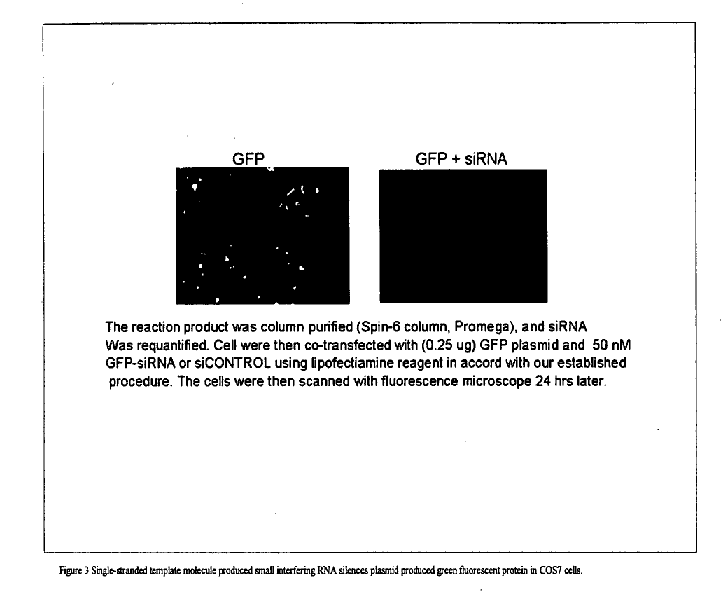

The present invention provides methods for the synthesis of RNA, DNA, or RNA/DNA hybrid molecules via a Single-stranded Template molecule (FIG. 1, FIG. 1A). The molecule is comprised of a single nucleic acid molecule (FIG. 1, FIG. 1A, and FIG. 1B) containing a first linker 6, first production sequence 8, first promoter complement sequence 10, first loop sequence 12, first promoter sequence 14, spacer sequence 16, second promoter sequence 18, production complement sequence 20, second loop sequence 22, second production sequence 23, second promoter complement sequence 24 and second linker 26. The primary nucleic acid molecule is allowed to fold and anneal to itself (FIG. 1, FIG. 1A) to form a functional partial double, partial single stranded molecule (i.e. Single-stranded Template molecule, FIG. 1). This novel molecule can be utilized to produce RNA, DNA, or RNA/DNA hybrid molecules of desired length and sequence in a single reaction vessel, in vitro, ex vivo, or in vivo.

The term “spacer sequence” 16 refers to any number of nucleotides in a sequence that allows for efficient polymerase enzyme activity or other functions (e.g. co-activation).

The term “promoter complement sequence” 10,24 refers to any number of nucleotides in a sequence that is complementary to the “promoter sequence” 14, and 18 respectively. This sequence is provided to form the “complete promoter” 10,14 and 24,18 respectively which collectively allow for the formation of the final functional form of the Single-stranded Template molecule and also acts to enhance the binding and activity of a corresponding polymerase. The two complete promoters may be of same or different sequence and thus may promote the activity of the same or different polymerase enzymes.

The term “promoter sequence” 14,18 refers to any number of nucleotides in a sequence that allows for the binding a polymerase and the synthesis of the products from the “production sequence” 8,23.

The term “production sequence” 8,23 refers to any number of nucleotides in a sequence that acts as a template for the partial or complete synthesis of the DNA, RNA, or hybrid DNA/RNA products; that can be either single stranded or duplexes (i.e. products).

The term “linker” 6, 26 refers to the position at which one Single-stranded Template molecule can be connected to another.

The term “loop sequence” 12,22 refers to a sequence of any number of nucleotides which is non-complementary to itself which allows for the formation of a duplex upstream and downstream from the “loop sequence.”

The term “production sequence complement” 20 refers to a sequence of any number of nucleotides which is complementary in sequence to the second “production sequence.”

In one embodiment the method comprises a production sequence which contains a sequence of any length enabling the production of complementary product that is complementary to itself and contains a “loop sequence” that facilitates the folding and self annealing and duplex formation thus forming double stranded DNA, or RNA, wherein each promoter drives the production of the same or distinct double stranded DNA, or RNA molecules.

The Single-stranded Template molecule(s) or its product(s) can be delivered in vitro, ex vivo, or in vivo, by incubation, direct injection, transfection, electroporation, transdermally, or orally.

The Single-stranded Template molecule can be delivered by the above mentioned methods for synthesis in vitro (i.e. inside or outside the cell), ex vivo, or in vivo.

The Single-stranded Template molecule can be synthesized in vitro (i.e. inside or outside of the cell), ex vivo, or in vivo, by incorporation of Single-stranded Template molecule into a host genome or by integration of a gene that when transcribed by an endogenous or exogenous polymerase would produce a Single-stranded Template molecule in vitro (i.e. inside or outside the cell), ex vivo, or in vivo.

The product can be synthesized by incorporation of Single-stranded Template molecule into a delivery vector. A delivery vector can be nothing, virus, bacteriophage, plasmid, liposome, exogenously delivered cells, re-engineered host cell, artificial chromosome, extrachromosomal array, carrier protein, bacteria, fungus, protozoa, plant cell, or other organism.

The product can also be synthesized by the inclusion of the necessary polymerizing enzymes exogenously, and/or endogenously produced via the delivery vector containing both the Single-stranded Template molecule for the endogenous polymerizing enzyme, or the necessary enzymes can be provided by a separate vector or added exogenously.

The production sequence can code for sequences that allow for integration into vectors, artificial chromosomes, or host genome.

In one of the preferred embodiment of the invention the production sequences are such that they are complementary to each other and can be used for gene silencing via RNA interference.

The present invention may entail the use of the products of the production sequence to silence genes that may be responsible for the maintenance of a cancerous state. A gene that is over expressed in cancerous cells or an inappropriately expressed cancer linked gene may be targeted to inhibit the cancerous growth.

The present invention as disclosed herein may involve the introduction of the Single-stranded Template molecule into host cells to render the host cell less susceptible to infection. Such methods may include targeting a gene or set of genes that are necessary for the infecting agent's survival or replication within the host cell.

The present invention can also be used to temporally silence a gene of interest to assay for its function at a certain developmental stage or age of the cell or organism. Since genes are regulated both temporally and spatially the delineation of their role in a temporal fashion would be used to assay for the function of a gene temporally.

In another such embodiment of the present invention, the products may remain single stranded and act as antisense mediated silencing agents.

The products of the production sequences can be used to silence a variety of genes of many origins including viral, bacterial, fungal, plant, protozoa, yeast, insect, animal, or mammalian cell genes by forming interfering RNA, DNA antisense, ribozymes, deoxyribozymes or other interfering nucleic acid molecule. The products may encode a protein that has functions including or beyond gene silencing.

Another preferred embodiment is the production of kits for the purpose of gene silencing via RNA, DNA or both, as well as the production of nucleic acid molecules of a specific sequence for any use (e.g. ribozymes and deoxyribozymes). A kit would include all of the necessary reagents for transcription of nucleic acids and would be performed in a single transcription vessel. This mix would include Single-stranded Template molecule(s) of a desired length and sequence as describe above, the corresponding enzyme(s) that would act at the promoter(s) contained within the Single-stranded Template molecule(s) (e.g. T7 polymerase, SP6 polymerase), buffers (e.g. Tris-HCl at pH 8.0, and EDTA) and enzyme transcription buffer (e.g. Tris-HCl at pH 7.9, MgCl2, DTT, NaCl and spermidine), nucleic acid tri-phosphates (NTPs), pyrophosphatase, and RNAase inhibitor, DNAase inhibitor, or both. The mix would then be incubated at the temperature appropriate for polymerization (e.g. 37.5° C. for 2 h). Nucleic acid sequences generated (i.e. products) can then be annealed after stopping the reaction by heating to a high temperature (e.g. 95° C. for 5 min) followed by an annealing temperature (e.g. 1 h at 37.5° C.) to obtain the crude products (e.g. small interfering double-stranded RNA). The mixture can then be further purified by nucleic acid precipitation (e.g. sodium acetate solution at pH 5.2, and then ethanol, dried and resuspended in water). Then the nucleic acid products can be further purified with enzymes (e.g. RNAse T1) and gel extraction methods. The final products can then be used for various procedures (e.g. gene silencing, genetic screening, transfection of any cell type, gene therapy).

The present invention may be used for the production of kits for the purpose of gene silencing via RNA, DNA or both, as well as the production of nucleic acid molecules of a specific sequence for any use (e.g. ribozymes and deoxyribozymes). A kit would include all of the necessary reagents for transcription of nucleic acids intracellularly, utilizing host cell enzymes and reagents for the production of nucleic acid molecules of a specific length and sequence. This mix would include Single-stranded template molecule(s) of a desired length and sequence as describe above, the corresponding enzyme(s) (if not provided by the host cell). Next the mix would then be processed to allow proper conditions for incubation/injection with the host cells/organisms (e.g. resuspended in normal saline, or with liposomal transfection reagents) and allowed to induce intracellular production of nucleic acid sequences (i.e. products) which can then be used for various procedures (e.g. gene silencing, gene screening, transfection of any cell type, gene therapy).

As disclosed herein, the production sequence can also code for nucleotide enzymes such as deoxyribozymes and ribozymes that can modify RNA to perform a variety of functions including gene silencing.

The production sequence can produce deoxyribozymes, and ribozymes which can act as RNA replicases and produce double stranded RNA for initiation of RNA interference or other functions. Accordingly the produced ribozymes and deoxyribozymes can be used in any cell type in any organism.

The present invention may entail the use of the Single-stranded Template molecule to produce single stranded primers for use by exogenous or endogenous provided enzymes. These primers, or any other types of oligonucleotides, for use by exogenous and/or endogenous enzymes can be produced by the Single-stranded Template molecule in any organism in vitro, ex vivo, or in vivo.

As disclosed herein the present invention may include methods wherein each of two production sequences of the Single-stranded Template molecule codes functionally distinct products, for example, one of the production sequences encodes a deoxyribozyme or ribozyme and the other codes for a hairpin molecule. These molecules can be designed to interact with themselves or endogenous or exogenous enzymes or other molecules. Moreover a likely modification of this embodiment can include a method wherein one or both of the produced ribozymes or deoxyribozymes target its Single-stranded Template of origin or another Single-stranded Template or products of the Single-stranded Template origin or other Single-stranded Templates.

Yet another preferred embodiment of the present invention is a method wherein the production sequences are used to induce translational suppression of protein synthesis by encoding products such as microRNAs and other interfering RNAs or DNAs.

Another alternative embodiment of the present invention may entail one where the Single-stranded Template molecule, and/or its produced sequences, and/or the necessary reagents, and/or the necessary enzymes corresponding to the promoters/primers on the Single-stranded Template molecule, and/or other necessary reagents and molecules needed for production of the products of the production sequence can be delivered via the skin, blood, gastrointestinal tract, eye drops, mucous membrane transfer gels, inhalants, intramuscular injections, intra-tissue implants, tissue/blood grafts, subcutaneous injections, as a contact dust, as a contact liquid, or in aerosol form.

Another preferred embodiment is one where the Single-stranded Template molecule, and/or its produced sequences, and/or the necessary reagents, and/or the necessary enzymes corresponding to the promoters/primers on the Single-stranded Template molecule, and/or other necessary reagents and molecules needed for production of the products of the production sequence can be delivered via the skin, blood, gastrointestinal tract, eye drops, mucous membrane transfer gels, inhalants, intramuscular injections, intra-tissue implants, tissue/blood grafts, subcutaneous injections, as a contact dust, as a contact liquid, or in aerosol form and used as antimicrobial/antiviral agents by targeting essential genes of bacterial, fungal, yeast, amoeba, plant, protozoan, insect, mammalian, or animal cells and/or viruses, for gene silencing by the products.

The present invention may entail a method wherein the Single-stranded Template molecule, and/or its produced sequences, and/or the necessary reagents, and/or the necessary enzymes corresponding to the promoters/primers on the Single-stranded Template molecule, and/or other necessary reagents and molecules needed for production of the products of the production sequence can be delivered via the skin, blood, gastrointestinal tract, eye drops, mucous membrane transfer gels, inhalants, intramuscular injections, intra-tissue implants, tissue/blood grafts, subcutaneous injections, as a contact dust, as a contact liquid, in aerosol form and used as an anticancer cell agent by targeting essential genes for cancer cell proliferation, and survival, for gene silencing by the products.

In still another preferred embodiment Single-stranded Template molecules or products can be utilized for high-throughput genetic screening assaying for gene function, target validation, biological pathway delineations or search for a desired phenotype.

The following examples are meant to be illustrative of the present invention; however, the practice of the invention is not limited or restricted in any way by them.

Single-stranded Template molecule directed T7 synthesis of small interfering RNA

The following Single-stranded Template molecule, designed to produce small Interfering RNA to silence green fluorescent protein, was obtained in desalted DNA oligonucleotide form: 5′-CGG CAA GCT GAC CCT GAA GTT CTA TAG TGA GTC GTA TTA TGT TCT C TAA TAC GAC TCA CTA TAG TGT TCT C TAA TAC GAC TCA CTA TAG CAA GCT GAC CCT GAA GTT CAT AAA AAA A ATG AAC TTC AGG GTC AGC TTG CTA TAG TGA GTC GTA TTA-3′. The Single-stranded Template molecule was reconstituted in a salt buffer designed to promote annealing (e.g. 10 mM Tris-HCl pH 7.5, 50 mM NaCl) and the mixture was heated to 95° C. for 5 minutes and then allowed to cool to room temperature for 2 hours. Single-stranded Template directed T7 driven transcription was performed according to previously described reports. Briefly, the following were added into a single vessel: transcription buffer (42 mM Tris-HCl pH 7.9, 11 mM NaCl, 4.5 mM MgCl2, 2.5 mM spermidine, and 11 mM DTT), 0.15 units yeast pyrophosphatase, 2 mM rNTP, 40 units RNase inhibiting peptide and 100 units T7 RNA polymerase, 200 pmol self-annealed Single-stranded Template molecule. Next, mix was incubated at 37° C. for 1 hour and 30 minutes, after which 1 unit of DNAse-I was added and then incubated at 37° C. for 30 min to remove DNA opposed template. Generated RNA formed sense and antisense strands of a small interfering RNA duplex designed to silence green fluorescent protein. The siRNA-GFP was precipitated by addition of ice cold 0.1 volumes of 3M sodium acetate (pH 5.1), and 1 volume of isopropanol and allowed to incubate for 10 minutes on ice. Next, siRNA-GFP was centrifuged at −20° C. at max speed (10K×g) for 30 minutes. The pellet was washed twice with 75% ethanol and dried, and then resuspended in pure water by heating at 55° C. for 10 minutes. The siRNA-GFP was then frozen at −80° C. until use.

Analysis of Single-Stranded Template Produced RNA

Single-stranded Template produced siRNA was analyzed by ethidium bromide staining on an agarose gel (after electrophoresis) in comparison with dsRNA marker. As expected the produced siRNA-GFP was of expected size, migrating similarly as the dsRNA marker of equal size. To further analyze the efficacy of the siRNA-GFP, COS7 cells were co-transfected with a plasmid expressing green fluorescent protein and small interfering RNA produced by the Single-stranded Template molecule designed to

What is claimed is:

1. A method of producing RNA, DNA, or hybrid RNA/DNA molecules having a defined length and sequence comprising: providing a primary single-stranded nucleic acid molecule containing variable length domains in this order: first linker sequence, first production sequence, first promoter complement sequence, first loop sequence, first promoter sequence, spacer sequence, second promoter sequence, second production complement sequence, second loop sequence, second production sequence, second promoter complement sequence, and second linker sequence. The promoters, promoter complements, loop, linkers, sequences can be of heterogeneous or homogeneous sequence, wherein the promoter complement sequences are complementary to a corresponding promoter sequence, and wherein the production complement sequence is complementary to a production sequence, enabling the formation of the loop structures. Additionally multiply primary single-stranded nucleic acid molecules containing the same domains as described above, either of homogenous or heterogeneous sequence may be linked at the linker sequences to form larger Single-stranded Template molecules. This Single-stranded Template molecule is then allowed to anneal and fold on itself to form a partial double, partial single stranded nucleic acid template, wherein an endogenous or exogenously provided polymerase(s) is(are) used to drive the production of RNA, DNA, or RNA/DNA hybrids, in vitro, ex vivo, or in vivo.

2. A method according to claim 1 wherein the spacer sequence can be zero to any number of base pairs. The spacer sequence can be a functional promoter element, promoter modifying element, or a nucleic acid sequence that simply links (i.e. linker sequence) other nucleic acid sequences together.

3. A method according to claim 1 wherein the promoter complement consists of any sequence that is complementary to its corresponding promoter sequence. This complementary sequence may or may not be a functional promoter element or a promoter modifying element.

4. A method according to claim 1 wherein the promoter consists of any sequence modulating the binding and subsequent initiation of polymerization of the product as read from the production sequence by any polymerizing enzyme, or modifiers of polymerizing enzymes.

5. A method according to claim 1 wherein the production sequence contains any sequence of any length enabling the production of an RNA, DNA, or RNA/DNA hybrid product.

6. A method according to claim 5 wherein the production sequence contains any sequence of any length, enabling the production of complementary product that is complementary to itself.

7. A method according to claim 1 wherein the Single-stranded Template molecule or its products is delivered in vitro, ex vivo, or in vivo, by direct injection, transfection, electroporation, transdermally, orally, liquid, dust, aerosol, gel, cream, or transfer solid.

8. A method according to claim 1 wherein the production sequence codes for a self-annealing RNA duplex having a defined length and sequence comprising: providing a primary single-stranded RNA to generate an RNA of defined length and sequence which is self-complementary over at least a portion of its length, and self-annealing thus forming a hairpin RNA duplex and used for gene silencing.

9. A method according to claim 1 wherein products are synthesized by in vitro or by in vivo transcription.

10. A method according to claim 1 wherein products are synthesized by incorporation of Single-stranded Template molecule host genome or into a delivery vector, wherein the delivery vector can be a virus, bacteriophage, plasmid, liposome, exogenous cell, re-engineered host cell, artificial chromosome, extrachromosomal array, carrier protein, carrier compound, or artificial chromosome and used in any cell type (e.g. bacterial, fungal, plant, protozoan, animal, insect, mammalian), or any virus type.

11. A method according to claim 1 wherein the polymerizing enzymes necessary for Single-stranded Template molecule synthesis of products are delivered with the Single-stranded Template molecules.

12. A method according to claim 1 wherein the polymerizing enzymes necessary for Single-stranded Template molecule synthesis of products are those contained within the host organism or genome, provided by organism associated flora, or provided upon host infection by virus or organism.

13. A method according to claim 1 wherein the production sequences produce complementary products of RNA, DNA or both RNA and DNA that will be used to form duplexes that can be used to sequence-specifically silence genes in any cell type (e.g. bacterial, fungal, plant, protozoan, animal, insect, mammalian), or any virus type.

14. A method according to claim 1 wherein the Single-stranded Template molecule synthesized single-stranded products can be used for gene silencing.

15. A method according to claim 5 wherein the produced RNA or DNA duplexes can be used to silence a target gene wherein the target gene to be silenced is that of any cell type (e.g. bacterial, fungal, plant, protozoan, animal, insect, mammalian), or any virus type.

16. A method according to claim 1 wherein the production sequence codes for ribozymes or deoxyribozymes that can be used in any cell type (e.g. bacterial, fungal, plant, protozoan, animal, insect, mammalian), or any virus type.

17. A method according to claim 1 wherein the production sequences and/or products is/are used to induce translational suppression of protein synthesis by encoding product such as microRNAs and other interfering RNAs or DNAs.

18. A method according to claim 1 wherein the Single-stranded Template molecule, and/or its produced sequences, and/or the necessary reagents, and/or the necessary enzymes corresponding to the promoters/primers on the Single-stranded Template molecule, and/or other necessary reagents and molecules needed for production of the products of the production sequence can be delivered via the skin, blood, gastrointestinal tract, eye drops, mucous membrane transfer gels, inhalants, intramuscular injections, intra-tissue implants, tissue/blood grafts, subcutaneous injections, as a contact dust, as a contact liquid, in aerosol form, via stem cells, via genetically engineered cancer cells, via genetically engineered patient-harvested cells, via genetically engineered normal cells, via genetically engineered bacteria, via genetically engineered viruses, via genetically engineer fungi, via genetically engineered protozoa, via genetically engineered plants or via genetically engineer bacteriophages, via carrier proteins, or carrier compounds, and used as a treatment for a disease or condition.

19. A method according to claim 1 wherein Single-stranded Template molecules can be utilized for high-throughput genetic screening assaying for gene function, protein expression and/or phenotype.

20. A method according the claim 1 wherein a kit can be designed for various uses (e.g. small interfering RNA synthesis, genetic screening, oligonucleotide synthesis, antisense gene silencing, microRNA synthesis for translational interference) wherein the Single-stranded Template molecule is a component.


SEQUENCE LISTING (1 sequence)

SEQ ID NO: 1 - DNA, 156 bp, Artificial
  This sequence is artificially produced and either used in vitro or incorporated for use in vivo.
cggcaagctgaccctgaagttctatagtgagtcgtattatgttctctaatacgactcactatagtgttctctaatacgactcactatagcaagctgaccctgaagttcataaaaaaaatgaacttcagggtcagcttgctatagtgagtcgtatta